(19) United States
(12) Patent Application Publication
(10) Pub. No.: US 2014/0093981 A1
(43) Pub. Date: Apr. 3, 2014

(54) METHOD FOR IMMOBILIZING ALBUMIN ON A SELF-ASSEMBLED MONOLAYER

(71) Applicant: PANASONIC CORPORATION, Osaka (JP)

(72) Inventor: Yukari HATAOKA, Kyoto (JP)

(73) Assignee: PANASONIC CORPORATION, Osaka (JP)

(21) Appl. No.: 14/095,928

(22) Filed: Dec. 3, 2013

Related U.S. Application Data
(63) Continuation of application No. PCT/JP2011/007238, filed on Dec. 22, 2011.

(30) Foreign Application Priority Data
Jul. 5, 2011 (JP) 2011-148917

(51) Int. Cl. C07K 14/76

(52) U.S. Cl. CPC C07K 14/76
USPC 436/501; 530/369; 530/362

(57) ABSTRACT

The method of the present disclosure is characterized by that one molecule of the amino acid is interposed between the self-assembled monolayer and the molecule of the albumin. For example, a method is provided for immobilizing albumin on a self-assembled monolayer, the method including the following steps (a) and (b) in this order: a step (a) of preparing a substrate including one molecule of an amino acid and the self-assembled monolayer and a step (b) of supplying the albumin to the substrate to form a peptide bond represented by a predetermined chemical formula as a result of reaction between the carboxyl group of the one molecule of the amino acid and the amino group of the albumin.

### CROSS REFERENCE TO RELATED APPLICATIONS

This is a continuation of International application No. PCT/JP2011/007238, with international filling date of Dec. 22, 2011, which claims priority of Japanese Patent Application No. 2011-148917, filed on Jul. 5, 2011, the contents of all of which are hereby incorporated by reference.

### BACKGROUND

1. Technical Field

The present disclosure relates to a method for immobilizing albumin on a self-assembled monolayer.

2. Description of the Background Art

A biosensor is used to detect or quantify a target substance contained in a sample. Some of biosensors include antigen to detect or quantify antibody.

When a sample containing antibody is supplied to the biosensor including albumin, which is an antigen, the antibody is detected or quantified by being bound to the albumin.

International publication No. WO00/04382 (referred to as “Patent Literature 1”) discloses a prior biosensor including antigen (see, e.g. Page 24 lines 23-26, Page 25 lines 3-20, Page 25 line 27-Page 26 line 13, and Page 26 lines 14-22, Page 28 lines 21-23, Page 32 lines 3-29, Page 35 line 21-Page 36 line 21of Patent Literature 1). FIG. 2 shows a biosensor disclosed in FIG. 7 of Patent Literature 1.

According to the description regarding FIG. 7 of Patent Literature 1, the biosensor is used for screening an activity of a biomolecule. The biosensor includes a monolayer 7, an affinity tag 8, an adaptor molecule 9, and a protein 10. The monolayer 7 is composed of a self-assembled monolayer represented by chemical formula: X-R-Y (see, Page 24 lines 23-26, Page 25 lines 3-20, Page 25 line 27-Page 26 line 13, and Page 26 lines 14-22 of Patent Literature 1). Examples of X, R, and Y are HS—, an alkane, and a carboxyl group, respectively (see, Page 25 lines 3-20, Page 25 lines 27-Page 26 line 13, and Page 28 lines 21-23 of Patent Literature 1).

### SUMMARY

In order to improve the detection sensitivity or the quantification accuracy of antigen, it is required to increase an amount of albumin to be immobilized on the biosensor.

The present inventor has discovered that the amount of the immobilized albumin per unit area was increased significantly by binding one molecule amino acid to a self-assembled monolayer and then immobilizing albumin. The present subject matter has been provided on the basis of the discovery.

The purpose of the present disclosure is to provide a method for increasing an amount of albumin to be immobilized on the self-assembled monolayer, and a sensor having the albumin immobilized in accordance with the same method.

The following items A1 to C6 are provided in the present disclosure.

A1. A method for immobilizing albumin on a self-assembled monolayer includes the following steps (a) and (b) in this order. The step (a) is a step of preparing a substrate including one molecule of an amino acid and the self-assembled monolayer. The one molecule of the amino acid is bound to the self-assembled monolayer through a peptide bond represented by the following chemical formula (I):

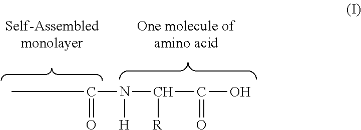

where R represents the side chain of the one molecule of the amino acid. The one molecular of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine. The step (b) is a step of supplying the albumin to the substrate to form a peptide bond represented by the following chemical formula (II) as a result of reaction between the carboxyl group of the one molecular of the amino acid and the amino group of the albumin:

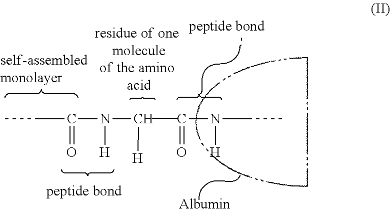

where R represents the side chain of the one molecule of the amino acid.

A2. In the method according to item A1, the step (a) may include the following steps (a1) and (a2). The step (a1) is a step of preparing a substrate including a self-assembled monolayer on the surface thereof, the self-assembled monolayer having a carboxyl acid at one end, and the step (a2) is a step of supplying the one molecule of the amino acid to form a peptide bond represented by the chemical formula (I) as a result of reaction between the carboxyl group of the one end of the self-assembled monolayer and the amino group of the one molecule of the amino acid.

A3. In the method according to item A1, the following step (ab) may be included between the step (a) and the step (b). The step (ab) is a step of activating the carboxyl group of the one molecule of the amino acid with a mixture of N-Hydroxysuccinimide and 1-ethyl-3-(3-dimethylaminopropyl)carbodiimide hydrochloride.

A4. In the method according to item A2, the following step (a1a) may be included between the step (a1) and the step (a2). The step (a1a) is a step of activating the carboxyl group of the self-assembled monolayer with a mixture of N-Hydroxysuccinimide and 1-ethyl-3-(3-dimethylaminopropyl)carbodiimide hydrochloride.

A5. In the method according to item A1, the chemical formula (II) may be represented by the following chemical formula (III):

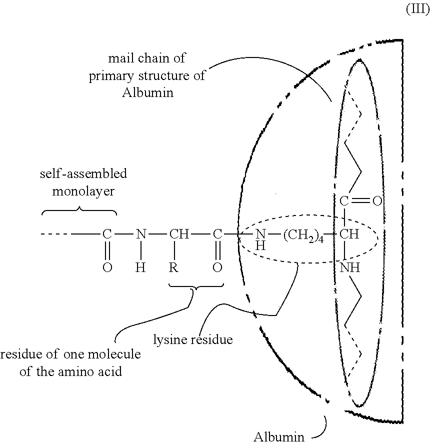

where R represents the side chain of the one molecule of the amino acid.

A6. In the method according to item A1, the one molecule of the amino acid may be selected from the group consisting of cysteine, lysine, histidine, phenylalanine, glycine, serine, alanine, glutamic acid, methionine, leucine, valine, tyrosine, isoleucine, threonine, asparagine, tryptophan and aspartic acid.

A7. In the method according to item A1, the one molecule of the amino acid may be selected from the group consisting of cysteine, lysine, histidine, phenylalanine, glycine, serine, alanine, glutamic acid, methionine and leucine.

A8. In the method according to item A1, the one molecule of the amino acid may be selected from the group consisting of cysteine, lysine, histidine, phenylalanine, glycine, serine, alanine, glutamic acid and methionine.

A9. In the method according to item A1, the one molecule of the amino acid may be selected from the group consisting of cysteine, lysine, histidine, phenylalanine, glycine and serine.

B 1. A sensor includes a self-assembled monolayer, one molecule of an amino acid, and albumin. The one molecule of the amino acid is interposed between the self-assembled monolayer and the albumin, and the albumin is bound to the self-assembled monolayer through two peptide bonds represented by the following chemical formula (II):

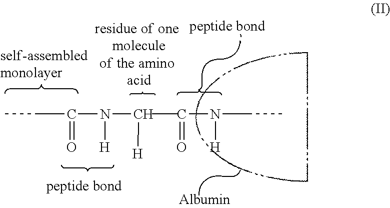

where R represents the side chain of the one molecule of the amino acid. The one molecular of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine.

B2. In the sensor according to item B1, the chemical formula (II) may be represented by the following chemical formula (III):

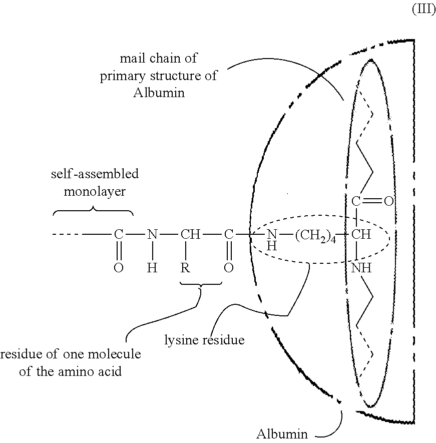

where R represents the side chain of the one molecule of the amino acid.

B3. In the sensor according to item B1, the one molecule of the amino acid may be selected from the group consisting of cysteine, lysine, histidine, phenylalanine, glycine, serine, alanine, glutamic acid, methionine, leucine, valine, tyrosine, isoleucine, threonine, asparagine, tryptophan and aspartic acid.

B4. In the sensor according to item B1, the one molecule of the amino acid may be selected from the group consisting of cysteine, lysine, histidine, phenylalanine, glycine, serine, alanine, glutamic acid, methionine and leucine.

B5. In the sensor according to item B1, the one molecule of the amino acid may be selected from the group consisting of cysteine, lysine, histidine, phenylalanine, glycine, serine, alanine, glutamic acid and methionine.

B6. In the sensor according to item B1, the one molecule of the amino acid may be selected from the group consisting of cysteine, lysine, histidine, phenylalanine, glycine and serine.

C1. A method for detecting or quantifying antibody contained in a sample with a sensor includes the following steps (a) to (c) in this order. The step (a) is a step of preparing the sensor including a self-assembled monolayer, one molecule of an amino acid, and albumin. The one molecule of the amino acid is interposed between the self-assembled monolayer and the albumin, and the albumin is bound to the self-assembled monolayer through two peptide bonds represented by the following chemical formula (II):

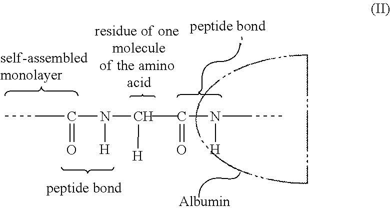

where R represents the side chain of the one molecule of the amino acid. The one molecular of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine. The step (b) is a step of supplying the sample to the sensor to bind the antibody to the albumin. The step (c) is a step of detecting the antibody bound in the step (b) or quantifying the antibody contained in the sample on the basis of the amount of the antibody bound in the step (b).

C2. In the method according to item C1, the chemical formula (II) may be represented by the following chemical formula (III):

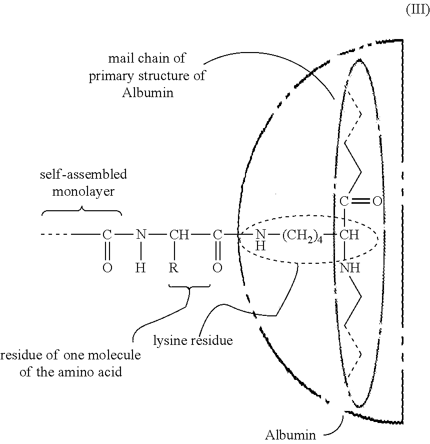

where R represents the side chain of the one molecule of the amino acid.

C3. In the method according to item C1, the one molecule of the amino acid may be selected from the group consisting of cysteine, lysine, histidine, phenylalanine, glycine, serine, alanine, glutamic acid, methionine, leucine, valine, tyrosine, isoleucine, threonine, asparagine, tryptophan and aspartic acid.

C4. In the method according to item C1, the one molecule of the amino acid may be selected from the group consisting of cysteine, lysine, histidine, phenylalanine, glycine, serine, alanine, glutamic acid, methionine and leucine.

C5. In the method according to item C1, the one molecule of the amino acid may be selected from the group consisting of cysteine, lysine, histidine, phenylalanine, glycine, serine, alanine, glutamic acid and methionine.

C6. In the method according to item C1, the one molecule of the amino acid may be selected from the group consisting of cysteine, lysine, histidine, phenylalanine, glycine and serine.

The present subject matter can significantly increase the amount of the albumin to be immobilized per unit area.

These and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 shows an exemplary schematic view of a method according to an embodiment of the present disclosure;

FIG. 2 corresponds to FIG. 7 of Patent Literature 1; and

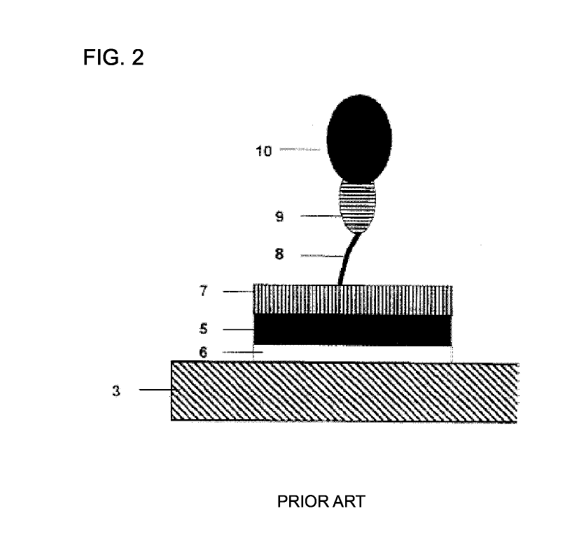

FIG. 3 shows a schematic view of a method according to the prior art.

### DETAILED DESCRIPTION

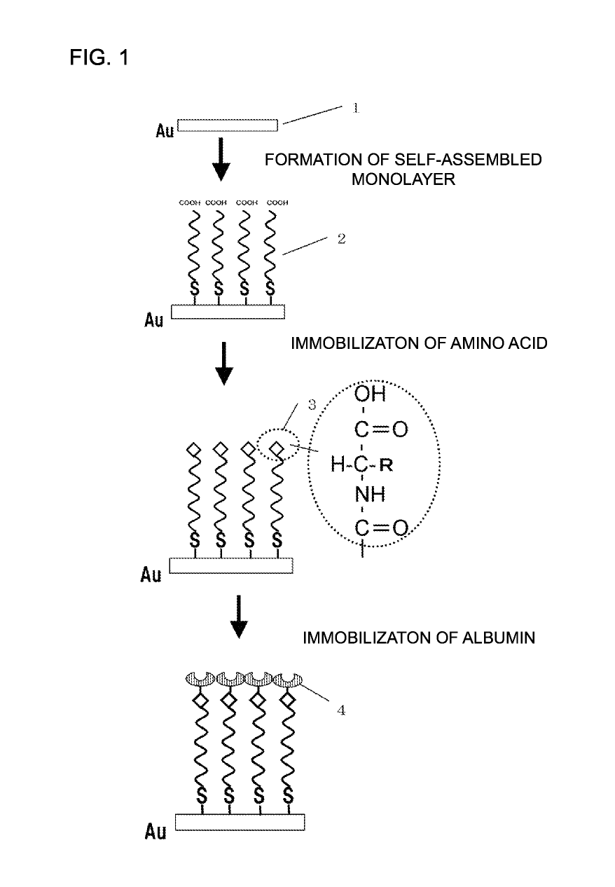

An embodiment of the present disclosure is explained below with reference to FIG. 1.

### Embodiment 1

FIG. 1 shows an exemplary method according to an embodiment of the present disclosure for immobilizing albumin on a self-assembled monolayer.

Preferably, a substrate 1 is a gold substrate. An example of the gold substrate is a substrate having gold uniformly on its surface. More particularly, the gold substrate may be a substrate having a gold film formed by a sputtering method on the surface of glass, plastic, or SiO₂.

First, the substrate 1 is immersed into a solvent containing an alkanethiol. Preferably, the substrate 1 is washed before it is immersed. The alkanethiol has a carboxyl group at the end thereof. It is preferable that the alkanethiol has the carbon number within the range from six to eighteen. Thus, a self-assembled monolayer 2 is formed on the substrate 1.

The preferred concentration of the alkanethiol is approximately 1 mM to 10 mM. The solvent is not limited to, as long as it dissolves the alkanethiol. An example of the preferred solvent is ethanol, dimethyl sulfoxide (hereinafter, referred to as “DMSO”), or dioxane. The preferred immersing period is approximately 12 to 48 hours.

Next, an amino acid 3 is supplied to the self-assembled monolayer 2. The carboxyl group (—COOH), which is located at the top end of the self-assembled monolayer 2, reacts with an amino group (—NH₂) of the amino acid 3 to form a peptide bond represented by the following the chemical formula (I):

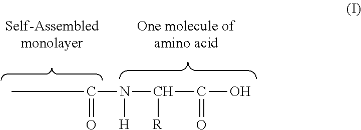

where R represents the side chain of the one molecule of the amino acid.

In the chemical formula (I), one molecule of the amino acid 3 binds to the self-assembled monolayer 2.

The amino acid 3 is selected from twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine In other words, in the chemical formula (I), R is the side chain of one amino acid selected from these twenty kinds of amino acids.

When the amino acid 3 is supplied to the self-assembled monolayer 2, two or more kinds of amino acids may be supplied simultaneously. In other words, when a solution containing the amino acid 3 is supplied to the self-assembled monolayer 2, the solution may contain two or more kinds of the amino acids 3. In light of uniform bind of the albumin to the amino acid 3, which is described later, it is preferred that the solution contains a sole kind of amino acid.

Subsequently, albumin 4 is supplied. The amino group of the N-terminus of the albumin 4 reacts with the carboxyl group of the amino acid 3. The amino group of the lysine included in the albumin also reacts with the carboxyl group of the amino acid 3. Thus, two peptide bonds represented by the following chemical formula (II) are formed to obtain a sensor:

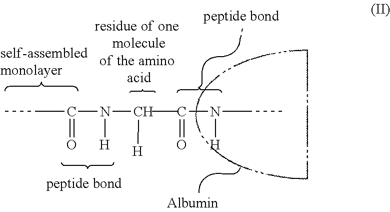

where R represents the side chain of the one molecule of the amino acid.

One molecule of the albumin 4 has only one N-terminus amino group whereas the one molecule of the albumin 4 has a lot of lysine groups having a free amine group. Accordingly, almost all of the chemical formula (II) is represented more specifically by the following chemical formula (III):

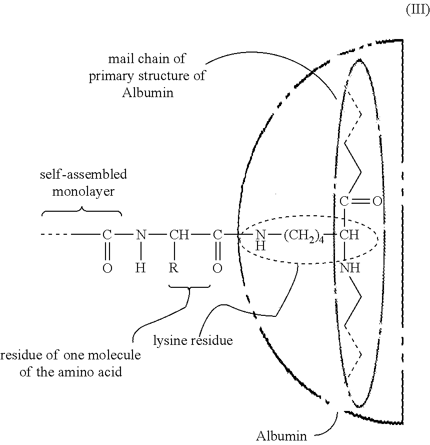

where R represents the side chain of the one molecule of the amino acid.

The sensor thus obtained is used for detecting or quantifying an antibody contained in the sample.

### EXAMPLES

The following examples and a comparative example describe the present disclosure in more detail. It is understood that the examples and embodiments described herein are for illustrative purposes only and that various modifications or changes in light thereof will be suggested to persons skilled in the art and are to be included within the spirit and purview of this application and scope of the appended claims

### COMPARATIVE EXAMPLE

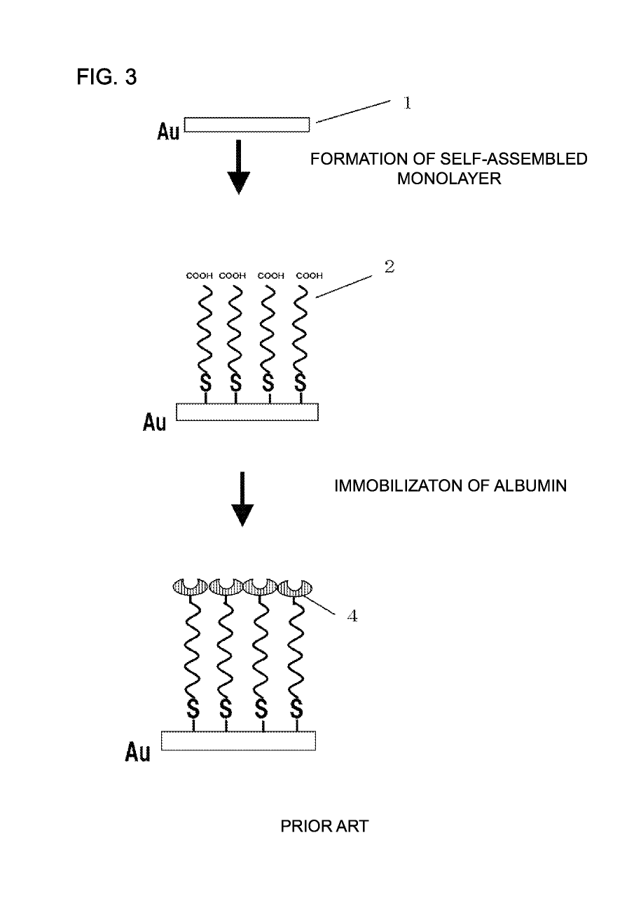

As shown in FIG. 3, albumin was bound directly with an amide coupling reaction to a carboxyl group located at the top end of self-assembled alkanethiol formed on the gold surface to immobilize the albumin. The procedure and the results were described below.

[Preparation of a Sample Solution]

A sample solution of 16-Mercaptohexadecanoic acid with final concentration of 10 mM was prepared. The solvent thereof was ethanol.

[Formation of a Self-Assembled Monolayer]

A gold substrate (available from GE healthcare company, BR-1004-05) with gold vapor-deposited on glass was used as a substrate 1. The substrate 1 was washed for ten minutes with a piranha solution containing concentrated sulfuric acid and 30% hydrogen peroxide water. The volume ratio of the concentrated sulfuric acid to the 30% hydrogen peroxide water contained in the piranha solution was 3:1.

Subsequently, the gold substrate was immersed in the sample solution for 18 hours to form a self-assembled monolayer on the surface of the gold substrate. Finally, the substrate 1 was washed with pure water and dried.

[Immobilization of Albumin]

Albumin was bound to the carboxyl acid group located at the top end of the 16-Mercaptohexadecanoic acid which formed the self-assembled monolayer to immobilize the albumin.

More particularly, the carboxyl group located at the top end of the 16-Mercaptohexadecanoic acid was activated with use of 35 microliters of a mixture of 0.1M NHS (N-Hydroxysuccinimide) and 0.4M EDC (1-ethyl-3-(3-dimethylaminopropyl)carbodiimide hydrochloride). Subsequently, thirty-five microliters of albumin (40 ug/ml) was added at the flow rate of five microliters/minute. Thus, the carboxyl group of the 16-Mercaptohexadecanoic acid was coupled with the amino group of the albumin.

### Example 1

Experiment was conducted similarly to the comparative example except that glycine was supplied as the one molecule of the amino acid between the formation of the self-assembled monolayer and the immobilization of the albumin. The procedure and the results are described below.

[Immobilization of Amino Acid (Glycine)]

Glycine was bound with the carboxyl group located at the top end of the 16-Mercaptohexadecanoic acid which formed the self-assembled monolayer 2 to immobilize the glycine.

Specifically, after the carboxyl group was activated similarly to the comparative example, 35 microliters of 0.1M glycine (pH:8.9) was added at the flow rate of 5 microliters/minute. Thus, the carboxyl group of 16-Mercaptohexadecanoic acid was coupled with the amino group of the glycine.

[Immobilization of Albumin]

Subsequently, albumin was bound to the carboxyl group of the glycine to immobilize albumin. More particularly, after the carboxyl group of the glycine was activated similarly to the above, 35 microliters of albumin (concentration: 250 micrograms/ml) was added at the flow rate of 5 microliters/minute. Thus, the carboxyl group was coupled with the amino group of the N-terminus of the albumin or the amino group of the lysine included in the albumin.

[Comparison of the Immobilization Amounts]

The immobilization amounts in the example 1 and in the comparative example were measured with use of an SPR device, Biacore 3000 (available from GE healthcare company).

The term “immobilization amount” means the amount of the albumin immobilized per unit area.

The ratio of the immobilization amount measured in the example 1 to the immobilization amount measured in the comparative example was approximately 14.4:1.

### Examples 2 to 20

Threonine, methionine, isoleucine, proline, serine, glutamine, asparagine, phenylalanine, tryptophan, cysteine, histidine, alanine, lysine, leucine, glutamic acid, valine, aspartic acid, argnine, and tyrosine were used instead of glycine to measure the respective immobilization amounts similarly to the example 1. These amino acids are twenty kinds of natural amino acid. Table 1 shows the measured immobilization amounts.

TABLE 1

| | | | |
|---|---|---|---|
| Example 11 | Cysteine | 19.49204 | |
| Example 14 | Lysine | 18.39829 | |
| Example 12 | Histidine | 16.81413 | |
| Example 9 | Phenylalanine | 15.16347 | |
| Example 1 | Glycine | 14.39286 | |
| Example 3 | Serine | 12.94221 | |
| Example 6 | Alanine | 12.7583 | |
| Example 13 | Glutamic acid | 11.42908 | |
| Example 8 | Methionine | 11.05119 | |
| Example 15 | Leucine | 10.66873 | |
| Example 20 | Valine | 8.958131 | |
| Example 16 | Threonine | 8.8923 | |
| Example 4 | Isoleucine | 8.802846 | |
| Example 2 | Tyrosine | 8.288947 | |
| Example 18 | Asparagine | 8.018876 | |
| Example 10 | Tryptophan | 7.88124 | |
| Example 17 | Aspartic acid | 6.962646 | |
| Example 19 | Argnine | 5.856666 | |
| Example 5 | Proline | 3.829463 | |
| Example 7 | Glutamine | 3.654396 | |
| | (None) | 1 | ←Comparative Example |

A skilled person would understand the following matters from Table 1.

When the twenty kinds of amino acids was used, the immobilization amounts increase, compared with the comparative example. Furthermore, the immobilization amount changes depending on the employed amino acid.

Cysteine, lysine, histidine, phenylalanine, glycine, serine, alanine, glutamic acid, methionine, leucine, valine, tyrosine, isoleucine, threonine, asparagine, tryptophan and aspartic acid are preferred, because each measured immobilization amount is five or more in a case where one amino acid selected from these amino acids is supplied.

Cysteine, lysine, histidine, phenylalanine, glycine, serine, alanine, glutamic acid, methionine and leucine are preferred, because each measured immobilization amount is ten or more in a case where one amino acid selected from these amino acids is supplied.

Cysteine, lysine, histidine, phenylalanine, glycine, serine, alanine, glutamic acid and methionine are more preferred, each measured immobilization amount is the average value (10.7) or more in a case where one amino acid selected from these amino acids is supplied.

Cysteine, lysine, histidine, phenylalanine, glycine and serine, are most preferred, because each measured immobilization amount is 1.2 times greater than the average value (12.9) in a case where one amino acid selected from these amino acids is supplied.

The present subject matter can achieve a significant increase of the amount of the albumin to be immobilized per unit area. This allows the sensitivity or the accuracy of the biosensor to be improved. The biosensor may be used for an inspection or a diagnosis which requires the detection or the quantification of the antibody contained in the living sample derived from a patient at a clinical practice.

While the subject matter has been described in detail, the foregoing description is in all aspects illustrative and not restrictive. It will be understood that numerous other modifications and variations can be devised without departing from the scope of the disclosure.

What is claimed is:

1. A method for immobilizing albumin on a self-assembled monolayer, the method comprising the following steps (a) and (b) in this order:
a step (a) of preparing a substrate comprising one molecule of an amino acid and the self-assembled monolayer; wherein:
the one molecule of the amino acid is bound to the self-assembled monolayer through a peptide bond represented by the following chemical formula (I): 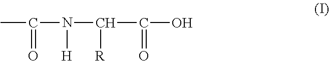
where R represents the side chain of the one molecule of the amino acid, and
the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine; and
a step (b) of supplying the albumin to the substrate to form a peptide bond represented by the following chemical formula (II) as a result of reaction between the carboxyl group of the one molecule of the amino acid and the amino group of the albumin: 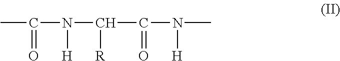
where R represents the side chain of the one molecule of the amino acid.

2. The method according to claim 1, wherein the step (a) comprises the following steps (a1) and (a2):
a step (a1) of preparing a substrate comprising a self-assembled monolayer on the surface thereof, the self-assembled monolayer having a carboxyl group at one end; and
a step (a2) of supplying the one molecule of the amino acid to form a peptide bond represented by the chemical formula (I) as a result of reaction between the carboxyl group of the one end of the self-assembled monolayer and the amino group of the one molecule of the amino acid.

3. The method according to claim 1, further comprising the following step (ab) between the step (a) and the step (b):
a step (ab) of activating the carboxyl group of the one molecule of the amino acid with a mixture of N-Hydroxysuccinimide and 1-ethyl-3-(3-dimethylaminopropyl)carbodiimide hydrochloride.

4. The method according to claim 2, further comprising the following step (a1a) between the step (a1) and the step (a2):
a step (a1a) of activating the carboxyl group of the self-assembled monolayer with a mixture of N-Hydroxysuccinimide and 1-ethyl-3-(3-dimethylaminopropyl)carbodiimide hydrochloride.

5. The method according to claim 1, wherein the chemical formula (II) is represented by the following chemical formula (III): 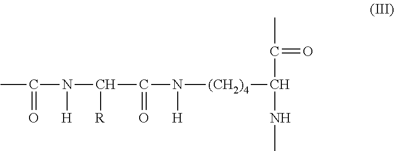
where R represents the side chain of the one molecule of the amino acid.

6. The method according to claim 1, wherein the one molecule of the amino acid is selected from the group consisting of cysteine, lysine, histidine, phenylalanine, glycine, serine, alanine, glutamic acid, methionine, leucine, valine, tyrosine, isoleucine, threonine, asparagine, tryptophan and aspartic acid.

7. The method according to claim 1, wherein the one molecule of the amino acid is selected from the group consisting of cysteine, lysine, histidine, phenylalanine, glycine, serine, alanine, glutamic acid, methionine and leucine.

8. The method according to claim 1, wherein the one molecule of the amino acid is selected from the group consisting of cysteine, lysine, histidine, phenylalanine, glycine, serine, alanine, glutamic acid and methionine.

9. The method according to claim 1, wherein the one molecule of the amino acid is selected from the group consisting of cysteine, lysine, histidine, phenylalanine, glycine and serine.

10. A sensor comprising a self-assembled monolayer, one molecule of an amino acid, and albumin, wherein:
the one molecule of the amino acid is interposed between the self-assembled monolayer and the albumin,
the albumin is bound to the self-assembled monolayer through two peptide bonds represented by the following chemical formula (II): 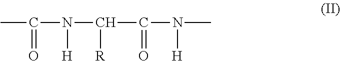
where R represents the side chain of the one molecule of the amino acid, and
the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine.

11. The sensor according to claim 10, wherein the chemical formula (II) is represented by the following chemical formula (III): 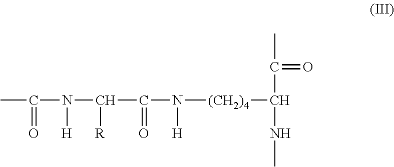
where R represents the side chain of the one molecule of the amino acid.

12. The sensor according to claim 10, wherein the one molecule of the amino acid is selected from the group consisting of cysteine, lysine, histidine, phenylalanine, glycine, serine, alanine, glutamic acid, methionine, leucine, valine, tyrosine, isoleucine, threonine, asparagine, tryptophan and aspartic acid.

13. The sensor according to claim 10, wherein the one molecule of the amino acid is selected from the group consisting of cysteine, lysine, histidine, phenylalanine, glycine, serine, alanine, glutamic acid, methionine and leucine.

14. The sensor according to claim 10, wherein the one molecule of the amino acid is selected from the group consisting of cysteine, lysine, histidine, phenylalanine, glycine, serine, alanine, glutamic acid and methionine.

15. The sensor according to claim 10, wherein the one molecule of the amino acid is selected from the group consisting of cysteine, lysine, histidine, phenylalanine, glycine and serine.

16. A method for detecting or quantifying antibody contained in a sample with a sensor, the method comprising the following steps (a) to (c) in this order:
a step (a) of preparing the sensor comprising a self-assembled monolayer, one molecule of an amino acid, and albumin, wherein:
the one molecule of the amino acid is interposed between the self-assembled monolayer and the albumin,
the albumin is bound to the self-assembled monolayer through two peptide bonds represented by the following chemical formula (II): 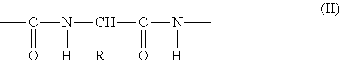
where R represents the side chain of the one molecule of the amino acid, and
the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine;
a step (b) of supplying the sample to the sensor to bind the antibody to the albumin; and
a step (c) of detecting the antibody bound in the step (b) or quantifying the antibody contained in the sample on the basis of the amount of the antibody bound in the step (b).

17. The method according to claim 16, wherein the chemical formula (II) is represented by the following chemical formula (III): 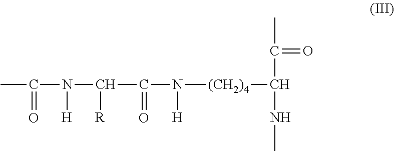
where R represents the side chain of the one molecule of the amino acid.

18. The method according to claim 16, wherein the one molecule of the amino acid is selected from the group consisting of cysteine, lysine, histidine, phenylalanine, glycine, serine, alanine, glutamic acid, methionine, leucine, valine, tyrosine, isoleucine, threonine, asparagine, tryptophan and aspartic acid.

19. The method according to claim 16, wherein the one molecule of the amino acid is selected from the group consisting of cysteine, lysine, histidine, phenylalanine, glycine, serine, alanine, glutamic acid, methionine and leucine.

20. The method according to claim 16, wherein the one molecule of the amino acid is selected from the group consisting of cysteine, lysine, histidine, phenylalanine, glycine, serine, alanine, glutamic acid and methionine.

21. The method according to claim 16, wherein the one molecule of the amino acid is selected from the group consisting of cysteine, lysine, histidine, phenylalanine, glycine and serine.
